(19) United States
(12) Patent Application Publication
(10) Pub. No.: US 2013/0150335 A1
(43) Pub. Date: Jun. 13, 2013

(54) PACLITAXEL/STEROIDAL COMPLEX

(71) Applicants: Yuling Liu, Beijing (CN); Xuejun Xia, Beijing (CN); Ruifang Guo, Beijing (CN); Pengxiao Zhang, Beijing (CN); Cuiping Zhou, Beijing (CN); Renyun Wang, Beijing (CN); Dujia Jin, Beijing (CN)

(72) Inventors: Yuling Liu, Beijing (CN); Xuejun Xia, Beijing (CN); Ruifang Guo, Beijing (CN); Pengxiao Zhang, Beijing (CN); Cuiping Zhou, Beijing (CN); Renyun Wang, Beijing (CN); Dujia Jin, Beijing (CN)

(73) Assignee: Institute of Mataria Medica, Chinese Academy of Medical Sciences, Beijing (CN)

(21) Appl. No.: 13/505,173

(22) PCT Filed: Oct. 28, 2010
(86) PCT No.: PCT/CN10/78202

§ 371 Date: Oct. 15, 2012

(30) Foreign Application Priority Data
Oct. 29, 2009 (CN) 200910236960.7

(51) Int. Cl. A61K 31/575; A61K 31/337

(52) U.S. Cl. CPC A61K 31/575; A61K 31/337
USPC 514/171

(57) ABSTRACT

A paclitaxel/steroid complex comprising paclitaxel and steroid is disclosed. The molar ratio of paclitaxel to steroid is 1:0.2˜4, preferably 1:0.25˜2. A process for the preparation thereof and the use thereof in the manufacture of submicron emulsion, dry emulsion, self-microemulsifying system are also disclosed.

### FIELD OF THE INVENTION

The present invention relates to a paclitaxel steroid complex and its preparation and use, and belongs to the technical field of pharmaceutical preparations.

### BACKGROUND OF THE INVENTION

Paclitaxel (paditaxel, Taxol) possesses an important anti-tumor activity, thus has been widely used in the treatment of ovarian and breast cancer, non-small cell lung cancer (NSCLC), head and neck carcinoma in clinical practice. Since it is barely soluble in water (0.006 μg/ml), the common paclitaxel injection formulation Taxol® presently used in clinical practice is prepared through dissolving 30 mg paclitaxel into 5 ml mixture of Cremopher EL (ethoxylate castor oil)/alcohol (50:50, V/V). Because of the large amount of Cremopher EL in the formulation, it tends to stimulate the release of histamine in vivo, resulting in severe allergic reactions. With regard to this, the following desensitization is required before clinical use: 10 mg oral dexamethasone is given 12 hours before treatment, and a second dose of 10 mg oral dexamethasone is given 6 hours before treatment, and 30 to 60 minutes before treatment, diphenhydramine, 20 mg, i.m., cimetidine, 300 mg, i.v. or ranitidine, 50 mg, i.v. is given. Despite these precautions, 5% to 30% of the patients may still experience allergies at different degrees in clinical practice. Furthermore, there may be physical stability issue once the concentrated paclitaxel solution solubilized by Cremopher EL/alcohol is diluted, for example, the drug may precipitate due to a low temperature or a long instilling time, thus patients' safety may be at risk.

Given the undesired effects of the paclitaxel injection formulation, pharmaceutical scientists both in China and worldwide have performed studies on a new formulation system since paclitaxel was launched more than 20 years ago, and technological strategies include formulations made by using cyclodextrin inclusion complex, liposome, polymeric micelle, nanoparticle and so on.

Although cyclodextrin inclusion complex could increase paclitaxel's solubility, cyclodextrin used at large quantities could cause severe renal toxicity; also the drug may precipitate once dilution is performed through water, therefore, this type of formulation has not been implemented in clinical practice so far.

Liposome has disadvantages including low entrapment efficiency, being prone to leakage if stored for a long time and precipitation after dilution through water, thus it is difficult to develop this type of formulation commercially and no product of this category is even though there has been on-going investment abroad for 20 years. The paclitaxel liposome (lipusu) freeze-dried power for injection includes 30 mg of the drug in each, its specification and dose for clinical use is identical to common injections available, and the efficacy is not significantly different, however, a preparation procedure is introduced and a pretreatment for desensitization is also needed, therefore, it is not technologically superior.

There are plenty of studies concerning paclitaxel polymeric micelle on-going both domestically and abroad, but its industrialization is limited by low drug loading, unstable quality after storage and requirement for lyophilization during storage. In the past years, research relating to polymeric micelle is developing very fast due to the sprouting of new polymer materials, however the medicinal safety of introducing large amounts of polymeric materials with completely new structures needs further evaluation.

The protein-bound paclitaxel nanoparticle injection (manufactured by Bioscience, Inc.) approved by FDA in 2005 is so far the most important new patented paclitaxel formulation all over the world, and it is designed to use human plasma albumin as a carrier and to prepare the paclitaxel formulation as protein-bound nanoparticles, which is made into freeze-dried power for injection after aseptic filtration, freezing and drying. Compared to common injections, the albumin-bound paclitaxel nanoparticle formulation for injection is superior in the following aspects: 1) this formulation is Cremopher EL free, thus allergic reaction is completely avoided, which makes it the only new paclitaxel formulation requiring no desensitization treatment worldwide; 2) due to its low toxicity and high tolerance, the clinical used dosage for patients is increased from 135˜175 mg/m² to 260 mg/m², thus resulting in a significantly better clinical efficacy than common paclitaxel injections. However, due to the large amount of the carrier, albumin, which is extremely expensive (up to 6200 Yuan for each injection), as well as its highly complicated and strict preparation procedures, the clinical use of albumin-bound paclitaxel nanoparticle is very limited.

The oil-in-water submicron emulsion is an emulsion of particles with an average diameter less than 600 nm obtained through homogeneous emulsification under high pressure using natural phospholipid as emulsifier after dissolving the medicine into the oil phase and the basis is the drug's lipotropy. Because inside the medicine there exists an inner oil phase, which avoids direct contact between water and air, thus overcoming the difficulty in preparing liquid formulations of drugs having low solubility and stability. Compared to liposome technology, submicron emulsion is more convenient to industrialize; and compared to albumin bound nanoparticle, an oil-in-water submicron emulsion has a lower manufacturing cost, could be sterilized at terminal, can be injected directly in clinical practice, does not tend to precipitate, and is safe and convenient to administrate. Therefore, there is a promising future to develop a new paclitaxel formulation using a submicron emulsion as carrier. Although both domestic and oversee scholars have done lots of experiments on paclitaxel submicron emulsions, the drug loading in the submicron emulsion manufactured through conventional procedures is under 0.02 mg/ml due to paclitaxel's low solubility in water as well as an extremely low solubility in oil; moreover, the medicine may transfer from the oil phase into the water phase during disinfection and storage, resulting in demulsification, stratification and concentration. Restricted by its low solubility in water, no paclitaxel submicron emulsion with high drug loading that is tolerant to sterilization under heat and pressure and stable through long-term storage has been developed in the world.

In order to improve paclitaxel's solubility in oil phase and ease the restriction from its physicochemical properties on the development of submicron emulsion, we performed preliminary studies on “Paclitaxel Liposome Complex” and “Paclitaxel Submicron Emulsion Using Liposome as Intermediate Carrier”, and applied for two patents: application No. CN200810168213.X, “Paclitaxel Liposome Complex” and application No. CN200810168212.5, “Paclitaxel Submicron Emulsion Using Liposome as Intermediate Carrier”.

For the paclitaxel liposome complex disclosed in patent application CN200810168213.X, natural egg yolk lecithin, granulesten and cholesterol were carefully chosen as the liposome material, and the proportion of paclitaxel and liposome material is 1:1˜19 by weight, i.e. the amount of liposome is up to 1˜19 times of that of paclitaxel (more specifically, for phospholipid, the mole ratio between paclitaxel and liposome is 1:1˜20; for cholesterol, it is 1:2.2˜20; for bile acids, it is 1:2.1˜40). The submicron emulsion formulation disclosed in patent application CN200810168212.5 adopts the paclitaxel liposome complex in patent application CN200810168213.X as the intermediate carrier.

The paclitaxel liposome complex is designed to improve the solubility of paclitaxel in oil and provide qualified intermediate carrier for subsequent manufacture of submicron emulsion. However, through further investigation, the following problems are identified in the technological protocol mentioned in patent applications CN200810168213.X and CN200810168212.5.

1. Although the drug solubility in oil could be significantly improved by using phospholipid to prepare the complex, the maximum is limited to 2 mg/ml, and the solubility in oil is not further increased by adding more phospholipid. Limited by low solubility in oil, the maximum drug loading is restricted to 0.5 mg/ml if submicron emulsion is prepared by using liposome complex as the intermediate carrier, and the entrapment efficiency is under 80%, there is obvious stratification after storage up to 6 months, thus it could not meet the requirements of medical treatment; with a drug loading up to 1.0 mg/ml, it could not form even emulsion.

2. Cholesterol could significantly improve the drug solubility in oil than phospholipid when used as the liposome material for the complex. However, cholesterol is a steroid, which could result in various disadvantages since its amount is 1˜19 times of that of paclitaxel: (1) overdose: a healthy adult intakes about 300 mg˜500 mg cholesterol each day (equivalent to the cholesterol in 1˜2 eggs), and one medicinal dose of paclitaxel is 300 mg, as for the cholesterol complex and its formulation involved in patent application CN200810168213.X, the cholesterol intake is about 300 mg˜5700 mg, with the highest dosage equivalent up to 19 egg yolks, which is significantly excessive and could lead to safety risk; (2) instability of the submicron emulsion prepared through long-term storage: if cholesterol complex is used as the intermediate carrier during submicron emulsion preparation, based on the medicinal formulation and specific paclitaxel concentration, higher the liposome material is used in the complex, more complex will be encapsulated inside the inner oil phase in the submicron emulsion. Restricted by the volume of the oil drop inside the oil phase and interface between oil and water, when the amount of complexes encapsulated exceeds that content that the oil phase and the interface between oil and water, part of the drug may be driven to the outer water phase, resulting in a low entrapment efficiency and instability of the submicron emulsion prepared. Through investigation of the submicron emulsion described in patent application CN200810168212.5, the entrapment efficiency is 65% to 85%, and the quality is essentially stable if stored for 6 months at 4° C.; However, there is obvious stratification when it is stored up to 12 months, the declared content drops and the paclitaxel impurity is significantly increased; (3) high manufacture cost: factors including large amounts of liposome used, solvent largely used during manufacture and long duration taken to evaporize the solvent, result in high manufacture cost, which disobeys the principle of pharmacological economy.

Therefore, on the basis of the two Chinese patent applications above, extensive testing and research have been conducted to select and optimize the lipid materials and their amounts, in order to develop a complex with lower quantities of lipid materials and lower cost, thereby improving the safety, effectiveness and quality control in clinical practice. The inventors of the present invention have found that among a large number of lipid materials, steroid is a preferable choice due to its strong capacity to complex with paclitaxel, which allows the use of a small quantity of steroid to solubilize the drug in oil to the maximum. Using the steroid complex described in present invention as the intermediate carrier to producing an oil-in-water submicron emulsion, because of the lower total amounts of the complex encapsulated, higher encapsulation efficiency as well as not being prone to demulsifciation during long-term storage, its physical and chemical stability are superior to those of the submicron emulsions disclosed in Chinese Patent Application CN200810168212.5. The paclitaxel steroid complex in the present invention has laid a solid foundation for the quality control and effectiveness as well as safety in clinical practice of the submicron emulsion developed therefrom.

### Contents of the Invention

An object of the present invention is to provide a paclitaxel/steroid complex composed of the paclitaxel and steroid lipid materials. The molar ratio of paclitaxel and steroid is 1:0.2˜4, preferably 1:0.25˜2, more preferably 1:0.33˜4.

In the paclitaxel/steroid complex of the present invention, the steroid lipid material is selected from the group consisting of a natural steroidal substance or a derivative thereof; said natural steroid is selected from the group consisting of cholesterol, 7-dehydrocholesterol (also known as 7-hydrogenated cholesterol), lanosterol, sitosterol, stigmasterol, sitosterolum, ergosterol, brassicasterol, mycosterol or oysters steroid; and its derivative is selected from the group consisting of cholic acid, deoxycholic acid and anthropodesoxycholic acid. A preferred steroid is selected from the group consisting of cholesterol, 7-dehydrogenation cholesterol and ergosterol, and a more preferred steroid is selected from cholesterol or ergosterol.

Said paclitaxel/steroid complex in the present invention may contain an antioxidant stabilizer. A preferred antioxidant stabilizer is selected from the group consisting of sodium bisulfite, sodium metabisulfite, vitamin C, EDTA and its salts, and vitamin E or its derivatives.

Another object of the present invention is to provide a method for the preparation of the subject paclitaxel/steroid complex, which can be prepared in accordance with the following Method 1 or 2.

Method 1 includes the following steps:

a. Mix paclitaxel and steroid in proportion, dissolve with an appropriate amount of an organic solvent, and then add an (i.e., any) antioxidant stabilizer;

b. Stir under a suitable temperature condition, remove the organic solvent, then vacuum dry.

Method 2 includes the following steps:

a. Dissolve paclitaxel and steroid separately in appropriate amounts of different organic solvents, mix, and add an (i.e., any) antioxidant stabilizer;

b. Stir under a suitable temperature condition, remove the organic solvent by rotary evaporation or spray drying, and then vacuum dry.

As described in the preparation method of the present invention, the organic solvents can be chosen from one or more of dichloromethane, ethanol, methanol, benzyl alcohol, acetone, ethyl acetate, tetrahydrofuran and tert-butanol; preferably one or more of ethanol, acetone, ethyl acetate and tetrahydrofuran. If several organic solvents are used, it is meant that a mixture of organic solvents is used.

In the preparation method of the invention, the removal of organic solvents can be achieved by rotary evaporation or spray drying.

In the preparation method of the present invention, “mix in proportion” refers to mix paclitaxel and steroid as mentioned above at a molar ratio of paclitaxel vs. steroid of 1:0.2˜4, preferably 1:0.25˜2, more preferably 1:0.33˜1; “an appropriate amount” in “an appropriate amount of organic solvents” refers to the amount of a mixture of organic solvents for dissolving paclitaxel and steroid as determined by those skilled in the art according to the conventional techniques. For example, the concentration of paclitaxel steroid complex in the solution is 0.5˜16 mg/ml, calculated as paclitaxel, preferably 1.0˜8.0 mg/ml; “the suitable temperature condition” refers to 25° C.-70° C., preferably 35-55° C., such as 25° C., 35° C., 45° C., 55° C. or 70° C.

In the preparation method of the present invention, the stirring and vacuum drying time can be determined by those skilled in the art in accordance with the conventional techniques, for instance stirring time can be 0.5-3.0 hours, e.g. 0.5 hour, 1.0 hour, 1.5 hours or 2.0 hours, and vacuum drying time can be 8-48 hours, e.g. 8 hours, 12 hours, 16 hours or 24 hours.

In the preparation method of the present invention, an appropriate amount of an antioxidant stabilizer may be added, and can be an amount commonly used in the preparation of liposome complex in the field, generally not more than 1% of the sum of paclitaxel and cholesterol (weight).

The present invention also provides use of the paclitaxel/steroid complex for the preparation of oil-in-water submicron emulsions, dry emulsions, self-microemulsifying systems or oral preparations. An oil-in-water submicron emulsion or dry emulsion is obtained by dissolving the paclitaxel/steroid complex in an oil phase, which can be administered by injection for cancer treatment. The preparation has the advantages of high drug loading, good stability as well as devoid of Cremopher EL in the formulation, and its safety is better than the commercial injection. A self-microemulsifying system can be obtained by dissolving paclitaxel/steroid complex in an oil phase, then adding an appropriate amount of surfactants (emulsifier) and cosurfactant (assisting emulsifying agent) and used for cancer treatment through injection, mucosal or oral administration. Oral preparations, in particular solid dosage forms such as capsules or tablets, can be obtained by adding excipients for pharmaceutical use in the subject paclitaxel steroid complex, intended for cancer therapy through oral administration, which has higher bioavailability.

The present invention also provides use of the paclitaxel/steroid complex of the present invention for the preparation of anti-cancer drugs, said cancer being selected from the group consisting of ovarian cancer, breast cancer, non-small cell lung cancer, head and neck cancer, and also for gastric or pancreatic cancer.

Unless specifically stated, the meanings of the science and terminology and titles as mentioned in the present invention comply with the general understanding those skilled in the field; and unless specifically pointed out, the substance to be used and its content or proportion, device, instrument, preparation conditions, etc. are well known by those skilled in the art or described in the present invention.

The paclitaxel/steroid complex in the present invention has the following advantages in particular:

1) Less lipid materials used and low preparation cost: a a complete combination of drug and lipid materials in complexes is a prerequisite for improvement of drug solubility in oil. Compared with paclitaxel complexes known in this field, including the paclitaxel complex as disclosed in Patent application CN200810168213.X, the molar ratio of drug vs. steroid lipid materials is 1:0.2˜4, preferably 1:0.25˜2, more preferably 1:0.33˜1.

The molecular weight of said steroid lipid materials in the present invention is 384.6˜414.7, thus if the molar ratio of drug to steroid is 1:0.2, the corresponding weight ratio is 1:0.09˜1:0.097; whereas if the molar ratio is 1:4, the corresponding weight ratio is 1:1.80˜1.94, that is the molar ratio of drug to steroid stands 1:0.2˜4 in the preferred range, the corresponding weight ratio is 1:0.09˜1.94. Accordingly if the molar ratio of drug to steroid stands 1:0.25˜2 in the preferred range, the corresponding weight ratio is 1:0.11˜0.97; whereas if the molar ratio of drug to steroid stands more preferably 1:0.33˜1, the corresponding weight ratio is 1:0.15˜0.49.

Compared with the contents as disclosed in Patent application CN200810168213.X, the steroid is used in the present invention as the lipid material, the weight ratio of drug to lipid reduces from 1:1˜19 to 1:0.09˜1.94 (preferably 1:0.11˜0.97), which decreases the amount of lipid materials, reduces the usage amount, improves the loading of paclitaxel in the complex (increased from 5%˜50% to 35%˜91.7%), and ensures paclitaxel to be fully combined in complex with the lipid material so that the maximum solubility of paclitaxel can be obtained in oil, which can meet the subsequent preparation of submicron emulsion. Increasing the amount of lipid materials can not continually improve the solubility of the drug in oil.

2) Less lipid materials to intake: take 300 mg of paclitaxel once as calculated, steroid intake should be controlled at 27 mg˜580 mg once, preferably 33 mg˜290 mg. Compared with 300 mg˜5700 mg as disclosed in Patent application CN200810168213.X, the steroid intake clearly represents much less lipid intake, which can reduce the health risk.

3) Improving the encapsulation efficiency and stability of submicron emulsion: The maximum amount of steroid in the complex of the present invention is only 1.94 times the amount of paclitaxel, preferably 0.97 times. If a compound is dissolved with vegetable oil to prepare an oil-in-water submicron emulsion, less total quantities of the compound within the oil phase can improve the encapsulation efficiency and physical and chemical stability in long-term storage. Comparative research has proved that when using the complex of the present invention as the intermediate carrier to prepare a submicron emulsion, the encapsulation efficiency can be kept at 90% above and the quality stability can be achieved at 4° C. for 12 months, whereas the encapsulation efficiency of the submicron emulsion as disclosed in Patent application CN200810168212.5 was at 65%-85% and layered phase separation appears if stored at 4° C. for 12 months and the extent of impurity increased significantly.

4) Enhancing the safety: Compared with the commercially available injection, the submicron emulsion formulation prepared with the paclitaxel/steroid complex as the intermediate carrier excludes Cremopher EL, which can avoid severe allergic reactions triggered by Cremopher EL, reduce animal toxicity and increase the tolerated dose.

Although the present invention has been fully described in connection with the description of figures and embodiments, it is to be noted that the invention included but not limited to these embodiments as well as the preparation method will become apparent to those skilled in the art. Moreover, according to the description, technicians in the field can equally substitute, combine, change or modify the compound as being included within the scope of the present invention.

### DESCRIPTION OF FIGURES

FIG. 1: DSC curve of the complex with different molar ratio and the physical mixture in Test Example 3.

FIG. 2: X-ray diffraction diagram in Test Example 6.

FIG. 3: Infrared spectrum in Test Example 7.

FIG. 4: FIG. 4-1: Ultraviolet spectrum in Test Example 8.

FIG. 4-2: Comparison of HPLC chromatographic peaks in Test Example 8 (peak 1: paclitaxel).

### EXAMPLES

### Preparation of Examples

### Example 1

### Paclitaxel Cholesterol Complex

Take 9.0 g of paclitaxel and 0.81 g of cholesterol in a rotary evaporator, dissolve with 3000 ml of acetone, mix at 40° C. for 1 hour, then remove the solvent by rotary evaporation method, vacuum drying at 40° C. for 12 hours.

### Example 2

### Paclitaxel Cholesterol Complex

Take 9.0 g of paclitaxel and 0.99 g of cholesterol in a rotary evaporator, dissolve with 3000 ml of tetrahydrofuran, mix at 25° C. for 1 hour, then remove the solvent by rotary evaporation method, vacuum drying at 25° C. for 12 hours.

### Example 3

### Paclitaxel Cholesterol Complex

Take 8.5 g of paclitaxel and 1.275 g of cholesterol in a triangle flask, dissolve with 3000 ml of tetrahydrofuran, mix at 45° C. for 1.5 hours, then remove the solvent by rotary evaporation method, vacuum drying at 45° C. for 16 hours.

### Example 4

### Paclitaxel Cholesterol Complex

Take 8.0 g of paclitaxel and 1.84 g of cholesterol in a triangle flask, dissolve with 3000 ml of acetone, mix at 35° C. for 1 hour, then remove the solvent by rotary evaporation method, vacuum drying at 35° C. for 10 hours.

### Example 5

### Paclitaxel Cholesterol Complex

Take 7.0 g of paclitaxel and 3.15 g of cholesterol in a triangle flask, dissolve with 32500 ml of acetone, mix at 50° C. for 2 hours, then remove the solvent by rotary evaporation method, vacuum drying at 50° C. for 15 hours.

### Example 6

### Paclitaxel Cholesterol Complex

Take 7.0 g of paclitaxel and 4.2 g of cholesterol in a triangle flask, dissolve with 5000 ml of ethyl acetate, mix at 55° C. for 1 hour, then remove the solvent by rotary evaporation method, vacuum drying at 40° C. for 15 hours.

### Example 7

### Paclitaxel Cholesterol Complex

Take 5.2 g of paclitaxel and 4.73 g of cholesterol in a rotary evaporator, dissolve with 3000 ml of dichloromethane, mix at 60° C. for 2.5 hours, then remove the solvent by rotary evaporation method, vacuum drying at 55° C. for 15 hours.

### Example 8

### Paclitaxel Cholesterol Complex

Take 5.2 g of paclitaxel, 4.85 g of cholesterol and 0.05 g of vitamin E in a rotary evaporator, dissolve with 4000 ml of tetrahydrofuran, mix at 40° C. for 1 hour, then remove the solvent by spray drying for 15 hours, vacuum drying at 55° C. for 15 hours.

### Example 9

### Paclitaxel Cholesterol Complex

Take 5.1 g of paclitaxel and 4.95 g of cholesterol in a rotary evaporator, dissolve with 5200 ml of a mixture of ethanol and tert-butanol, mix at 45° C. for 1 hour, then remove the solvent by rotary evaporation method, vacuum drying at 65° C. for 15 hours.

### Example 10

### Paclitaxel Cholesterol Complex

Take 5.0 g of paclitaxel and 7.51 g of cholesterol in a rotary evaporator, dissolve with 5200 ml of a mixture of ethanol and tert-butanol, mix at 45° C. for 1 hour, then remove the solvent by rotary evaporation method, vacuum drying at 65° C. for 15 hours.

### Example 11

### Paclitaxel Cholesterol Complex

Take 5.0 g of paclitaxel and 9.10 g of cholesterol in a rotary evaporator, dissolve with 5200 ml of a mixture of ethanol and tert-butanol, mix at 45° C. for 1 hour, then remove the solvent by rotary evaporation method, vacuum drying at 65° C. for 15 hours.

### Example 12

### Paclitaxel/7-Dehydrocholesterol Complex

Take 5 g of paclitaxel, add 0.55 g of 7-dehydrocholesterol in a rotary evaporator, dissolve with 500 ml of acetone, mix at 45° C. for 1 hour, then remove the solvent by rotary evaporation method, vacuum drying at 65° C. for 15 hours.

### Example 13

### Paclitaxel/7-Dehydrocholesterol Complex

Take 5 g of paclitaxel, add 4.85 g of 7-dehydrocholesterol in a rotary evaporator, dissolve with 500 ml of acetone, mix at 45° C. for 1 hour, then remove the solvent by rotary evaporation method, vacuum drying at 65° C. for 15 hours.

### Example 14

### Paclitaxel/7-Dehydrocholesterol Complex

Take 5 g of paclitaxel, add 9.70 g of 7-dehydrocholesterol in a rotary evaporator, dissolve with 500 ml of acetone, mix at 45° C. for 1 hour, then remove the solvent by rotary evaporation method, vacuum drying at 65° C. for 15 hours.

### Example 15

### Paclitaxel/Ergosterol Complex

Take 5 g of paclitaxel and 0.54 g of ergosterol in a rotary evaporator together, dissolve with 500 ml of acetone, mix at 45° C. for 1 hour, then remove the solvent by rotary evaporation method, vacuum drying at 65° C. for 15 hours.

### Example 16

### Paclitaxel/Ergosterol Complex

Take 5 g of paclitaxel and 4.83 g of ergosterol in a rotary evaporator, dissolve with 500 ml of acetone, mix at 45° C. for 1 hour, then remove the solvent by rotary evaporation method, vacuum drying at 65° C. for 15 hours.

### Example 17

### Paclitaxel/Ergosterol Complex

Take 5 g of paclitaxel and 9.10 g of ergosterol in a rotary evaporator, dissolve with 500 ml of acetone, mix at 45° C. for 1 hour, then remove the solvent by rotary evaporation method, vacuum drying at 65° C. for 15 hours.

### Example 18

### Paclitaxel/Cholic Acid Complex

Take 4 g of paclitaxel each in 5 portions, add 0.5 g, 2.0 g, 3.0 g, 4.6 g and 7.70 g of cholic acid separately in a rotary evaporator, dissolve with 1000 ml of acetone each, mix at 45° C. for 1 hour, then remove the solvent by rotary evaporation method, vacuum drying at 65° C. for 15 hours.

### Example 19

### Submicron Emulsion Prepared with Paclitaxel/Steroid Complex as the Intermediate Carrier

Dissolve 25 g of glycerol with approximate 720 ml of water for injection, heat to 40-60° C., add 15 g of refined soybean lecithin and 30 g of poloxamer (188) to a blender, obtain a homogeneous water phase, keep at its temperature; take a quantity of a paclitaxel/steroid complex from Example 1 to 18 (equivalent to 500 mg of paclitaxel), add 200 ml of soybean oil, heat to 40-60° C., obtain the oil phase. Mix the water phase slowly into the oil phase, stir to producing uniform colostrum. Transfer the colostrum to a homogenizer several times, adjusting pH to 4.0-6.0 with 0.1 mol/L of hydrochloric acid, collect all emulsion, sterilize at 115° C. for 30 mins, thereby obtaining a submicron emulsion with 0.5 mg/ml of drug loading. The average particle size was 125 nm-150 nm detected by Lazer particle size analyzer.

### Example 20

### Submicron Emulsion Prepared with Paclitaxel/Steroid Complex as the Intermediate Carrier

Dissolve 12.5 g of glycerol with approximate 300 ml of water for injection, heat to 50° C., add 7.5 g of refined egg yolk lecithin and 15 g of poloxamer (188) to a blender, obtain a homogenous water phase, keep at its temperature; take a quantity of a paclitaxel/steroid complex from Example 5, 13 and 16 (equivalent to 500 mg of paclitaxel), add 125 ml of a mixture containing soybean oil and MEDIUM CHAIN OIL by volume, heat to 50° C., obtain the oil phase. Mix the water phase slowly into the oil phase, stir at high speed to producing uniform colostrum. Transfer the colostrum to a homogenizer several times, adjusting pH to 4.0-6.0 with 0.1 mol/L of hydrochloric acid, add water to 500 ml, collect all emulsion, sterilize at 115° C. for 30 mins, therebye obtaining a submicron emulsion with 1.0 mg/ml of drug loading. The average particle size was 230 nm˜250 nm detected by Lazer particle size analyzer.

### Example 21

### Submicron Emulsion Prepared with Paclitaxel/Steroid Complex as the Intermediate Carrier

Dissolve 12.5 g of glycerol with approximate 300 ml of water for injection, heat to 55° C., add 7.5 g of refined soybean lecithin and 15 g of poloxamer (188) to a blender, obtain a homogenous water phase, keep at its temperature; take a quantity of a paclitaxel/steroid complex from Example 1 to 18 (equivalent to 600 mg of paclitaxel), add 125 ml of soybean oil, heat to 55° C., obtain the oil phase. Mix the water phase slowly into the oil phase, stir at high speed to producing uniform colostrum. Transfer the colostrum to a homogenizer several times, adjusting pH to 4.0-6.0 with 0.1 mol/L hydrochloric acid, add water to 500 ml, collect all emulsion, sterilize at 115° C. for 30 mins, thereby obtaining a submicron emulsion with 1.2 mg/ml of drug loading. The average particle size was 125 nm˜133 nm detected by Lazer particle size analyzer.

### Example 22

### Submicron Emulsion Prepared with Paclitaxel/Steroid Complex as the Intermediate Carrier

Dissolve 12.5 g of glycerol with approximate 300 ml of water for injection, heat to 60° C., add 7.5 g of refined soybean lecithin and 15 g of poloxamer (188) to a blender, stir at high speed to producing homogenous water phase, keep at its temperature; take a quantity of paclitaxel/steroid complex from Example 1 to 18 (equivalent to 1000 mg of paclitaxel) respectively, add 150 ml of soybean oil, heat to 60° C., obtain the oil phase. Mix the water phase slowly into the oil phase, stir at high speed to producing uniform colostrum. Transfer the colostrum quickly to a homogenizer several times, adjusting pH to 4.0-6.0 with 0.1 mol/L of hydrochloric acid, add water to 500 ml, collect all emulsion, sterilize at 115° C. for 30 mins, thereby obtaining a submicron emulsion with 2.0 mg/ml of drug loading. The average particle size was 128 nm˜141 nm detected by Lazer particle size analyzer.

### Example 23

### Dry Emulsion Prepared with Paclitaxel/Steroid Complex as the Intermediate Carrier

Take a quantity of a non-sterilized submicron emulsion from Example 20 to 22, dissolve with 3% (W/V) of mannitol, sterilze and filter through 0.22 μm of microporous membrane, freeze dry.

### Example 24

### Self-Microemulsion Prepared with Paclitaxel/Steroid Complex as the Intermediate Carrier

Take a quantity of paclitaxel/steroid complex (equivalent to 100 mg of paclitaxel) form Example 1-18 respectively, add 5 ml of MEDIUM CHAIN OIL, mix to dissolve, add 1.6 ml of PEG400 and 0.5 ml of Tween-80, mix well, thereby obtaining a uniform and transparent self-microemulsifying system.

Take 10 ml of the self-microemulsifying system as mentioned above, add 5% of glucose injection to 50 ml, and then obtain a microemulsion promptly with particle sizes less than 100 nm.

### Example 25

### Capsules Prepared with Paclitaxel/Steroid Complex as the Intermediate Carrier

Take a quantity of a paclitaxel/steroid complex (equivalent to 250 mg of paclitaxel) form Example 1-18 respectively into No. 2 hard capsules.

### Example 26

### Tablets Prepared with Paclitaxel/Steroid Complex as the Intermediate Carrier

Take a quantity of a paclitaxel/steroid complex (equivalent to 500 mg of paclitaxel) from Example 1-18 respectively, add 500 mg of microcrystalline cellulose, 500 mg of lactose, and an amount of magnesium stearate, mix well, adjust the punch to compressing 100-250 mg of tablets in specification.

### Test Examples

### Test Example 1

### Technical Solution Validation as Disclosed in China Patent application 200810168213.X

Carry out the preferred lipid materials and their amount as disclosed in China Patent application 200810168213.X, use soya bean lecithin and cholesterol as lipid materials, use the weight ratio of drug to lipid materials of 1:1.85˜18.5, prepare the reference complex, investigate the solubility in soybean oil (calculated as paclitaxel), and compare with uncombined free paclitaxel.

TABLE 1
Validation results from the complex as disclosed in
Patent application 200810168213.X

| | Amount ratio of paclitaxel/lipid | | |
|---|---|---|---|
| | materials | | Solubility in soybean oil |
| Complex | Molar ratio | Weight ratio | (mg/ml) |
| Paclitaxel/ | 1:2 | 1:1.85 | 0.68 |
| Phospholipids | 1:4 | 1:3.70 | 0.75 |
| | 1:6 | 1:5.55 | 2.12 |
| | 1:10 | 1:9.23 | 2.32 |
| | 1:20 | 1:18.46 | 2.25 |
| Paclitaxel/ | 1:4 | 1:1.82 | 9.04 |
| Cholesterol | 1:6 | 1:2.73 | 8.87 |
| | 1:10 | 1:4.53 | 9.15 |
| | 1:20 | 1:9.06 | 8.75 |
| | 1:40 | 1:18.12 | 8.69 |
| Paclitaxel | / | / | 0.27 |
| Reference | | | |

The results show that the amount of phospholipids was 5.55 times that of paclitaxel, solubility appeared to be stable and controlled at 2.12˜2.32 mg/ml; while the amount of cholesterol was 1.82˜18.12 times the weight of paclitaxel, the solubility was controlled at 8.75˜9.15 mg/ml. These results suggest that using cholesterol as the lipid material is superior to phospholipids in the aspect of improving the solubility of drug substance in oil and the amount of cholesterol is expected to be further decreased and optimized.

### Test Example 2

### Influencing Factors Analysis of Paclitaxel/Steroid Complex in Oil

Amount Ratio of Paclitaxel to Steroid

Take 4 g of paclitaxel, add cholesterol, 7-dehydrocholesterol and ergosterol respectively in accordance with the amount ratio as stated in Table 2 below, move to a rotary evaporator together, dissolve with 500 ml of acetone each, mix at 45° C. for 1 hour then remove the solvent by the rotary evaporation method, vacuum dry at 65° C. for 15 hours. Take a quantity of each complex formed, add 20 g of soybean oil for injection used, heat in water bath for 1 hour and shake, obtain saturated solutions. Filter the solutions, take a quantity of the successive filtrate, add absolute ethanol to volume, inject 20 μl into the liquid chromatography, using Kromasil-C18 (250 mm×4.6 mm, 5 μm) as the column, acetonitrile-water (54:46) as the mobile phase, with a flow rate at 1.0 ml/min and a detection wavelength at 230 nm, calculate the solubility of paclitaxel in vegetable oil, see results in the table below.

TABLE 2
Effect of amount ratio of paclitaxel and steroid on the
solubility of the complex in oil

| | Amount ratio of paclitaxel and | | Solubility |
|---|---|---|---|
| Steroid lipid | steroid | | in soybean |
| materials | Molar ratio | Weight ratio | oil (mg/ml) |
| Cholesterol | 5:1 (1:0.2) | 1:0.091 | 7.66 |
| | 4:1 (1:0.25) | 1:0.112 | 8.25 |
| | 3:1 (1:0.33) | 1:0.151 | 8.56 |
| | 2:1 (1:0.5) | 1:0.225 | 9.14 |
| | 1:1 | 1:0.453 | 9.49 |
| | 1:2 | 1:0.906 | 9.06 |
| | 1:4 | 1:1.813 | 9.04 |
| 7-dehydrocholesterol | 5:1 (1:0.2) | 1:0.090 | 7.47 |
| | 4:1 (1:0.25) | 1:0.113 | 7.93 |
| | 3:1 (1:0.33) | 1:0.150 | 8.77 |
| | 2:1 (1:0.5) | 1:0.225 | 9.21 |
| | 1:1 | 1:0.450 | 8.85 |
| | 1:2 | 1:0.901 | 8.69 |
| | 1:4 | 1:1.802 | 9.12 |
| Ergosterol | 5:1 (1:0.2) | 1:0.093 | 7.23 |
| | 4:1 (1:0.25) | 1:0.116 | 8.50 |
| | 3:1 (1:0.33) | 1:0.155 | 9.26 |
| | 2:1 (1:0.5) | 1:0.232 | 8.78 |
| | 1:1 | 1:0.465 | 8.59 |
| | 1:2 | 1:0.929 | 9.11 |
| | 1:4 | 1:1.858 | 8.73 |
| Paclitaxel reference | / | / | 0.27 |

The results show that the molar ratio of paclitaxel to steroid was 1:0.2˜4; that is when the maximum amount of steroid was less than 4 times the molar amount of paclitaxel, the maximum solubility of drug in oil was already obtained.

Paclitaxel Concentration

Using acetone as the solvent, the molar ratio of paclitaxel to cholesterol being 1:1, the reaction temperature at 35° C., the reaction time being 0.5 h, the effect of the paclitaxel concentration (16.0, 8.0, 4.0, 2.0, 1.0 and 0.5 mg/ml) on the complex preparation was investigated. The results show that the concentration of paclitaxel had no obvious effect on the solubility of the complex in oil, see the table below.

TABLE 3
Effect of paclitaxel concentration on solubility of
cholesterol complex in oil

| Paclitaxel concentration | Solubility in soybean oil |
|---|---|
| (mg/mL) | (mg/ml) |
| 16.0 | 8.89 |
| 8.0 | 9.21 |
| 4.0 | 9.58 |
| 2.0 | 9.47 |
| 1.0 | 9.39 |
| 0.5 | 8.44 |

### Test Example 3

### Paclitaxel Cholesterol Complex in DSC Curves

Test Samples:
Paclitaxel
Cholesterol
Paclitaxel cholesterol complex (complex as prepared in table 2 of Test Example 2 with a molar ratio of 4:1, 3:1, 2:1, 1:1, 1:2, 1:4);
Physical mixture of paclitaxel and cholesterol (take a quantity of paclitaxel and cholesterol with the same molar ratio, mix well, and use for comparative research)

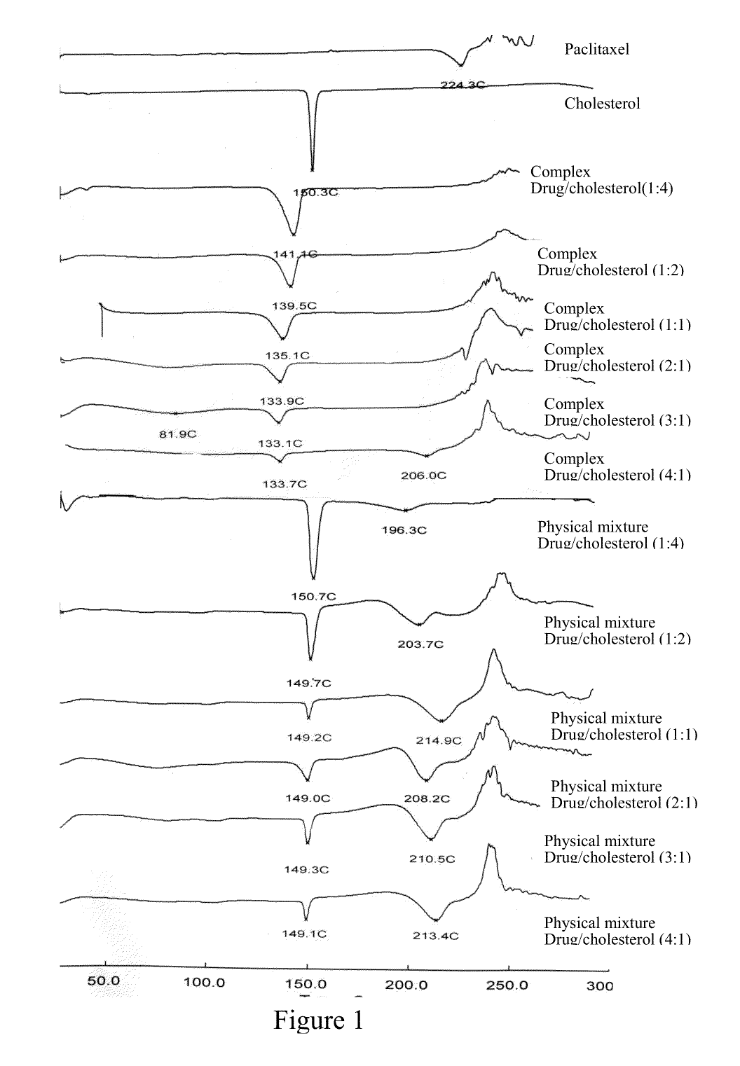

Method: carry out the differential scanning calorimetry (DSC) to determine the characteristics of DSC curves, temperature at 25˜300° C., with a heating rate of 10° C./min, nitrogen flow rate at 60 ml/min; the DSC curves are shown in FIG. 1.

Result: the endothermic melting peak of paclitaxel was at 225.7° C. and that of cholesterol was at 150.9° C. In the preparation of the physical mixture in different proportions, the endothermic characteristics of cholesterol did not change with the melting point at 149-150° C.; whereas the endothermic peak of paclitaxel had a little shift but its melting feature remained, it was just a simple physical mixture. The reason for the shift in the melting peak obtained with paclitaxel in the physical mixture was because the melting point of cholesterol is lower than that of paclitaxel; therefore after the melting of cholesterol, it affected the dispersed state of the drug, whichresulted in the change in the melting characteristics of paclitaxel.

In the preparation of the complex in different proportions, the melting characteristics of cholesterol clearly shifted, and the endothermic peak of paclitaxel disappeared completely, which indicates that both fully complexed with each other.

### Test Example 4

### Solubility Research on Paclitaxel Cholesterol Complex in Different Oil Phases

Take a quantity of the paclitaxel cholesterol complex as described in table 2 of Test Example 2, add a mixture of soybean oil for injection, MEDIUM CHAIN OIL and soybean oil/MEDIUM CHAIN OIL (1:1), heat to 60° C., mix well to dissolve, heat in the water bath at 60° C. for 1 hour, shaking constantly during the process, obtain a saturated solution. Filter the solution, take a quantity of the successive filtrate, dilute with absolute ethanol, carry out an HPLC analysis, calculate the solubility of complex in oil; take a quantity of paclitaxel, repeat the procedure. Compared the solubility of the complex with paclitaxel, the results have proved that the solubility of the drug in oil was improved when the drug was combined with cholesterol in different proportions, see the table below:

TABLE 4
Results of drug solubility in vegetable oil (mg/ml)

| | Drug/Cholesterol | | | | | | | |
|---|---|---|---|---|---|---|---|---|
| | 5:1 | 4:1 | 3:1 | 2:1 | 1:1 | 1:2 | 1:4 | Paclitaxel |
| Soybean | 7.66 | 8.25 | 8.56 | 9.14 | 9.49 | 9.06 | 9.04 | 0.27 |
| oil | | | | | | | | |
| MCO | 25.25 | 26.21 | 26.47 | 26.45 | 23.54 | 27.69 | 27.69 | 2.37 |
| Mixed | 15.51 | 13.87 | 14.02 | 16.56 | 15.50 | 16.89 | 16.89 | 1.36 |
| oil* | | | | | | | | |

Note:
mixed oil* was a mixture of soybean oil and MEDIUM CHAIN OIL (volume ratio of 1:1)

### Test Example 5

### Solubility of Paclitaxel Cholesterol Complex in n-Octanol

Take a quantity of the paclitaxel cholesterol complex described in table 2 into 50 ml of conical flask, add 10 g of n-octanol, shake at 25° C. in the thermostatic oscillator for 24 hours, obtain a saturated solution. Take 5 ml of the solution, filter by 0.45 μm of membrane, take an amount of the successive filtrate, dilute with absolute ethanol, carry out the ultraviolet spectrophotometry method, calculate the apparent solubility of the physical mixture and cholesterol in n-octanol, compared the results with paclitaxel. The results have proved that the solubility in n-octanol was improved when the drug was combined with cholesterol in different proportions, see the table below:

TABLE 5
Results of drug solubility in n-octanol (mg/ml, 25° C.)

| | Drug/Cholesterol | | | | | | |
|---|---|---|---|---|---|---|---|
| | 4:1 | 3:1 | 2:1 | 1:1 | 1:2 | 1:4 | Paclitaxel |
| Solubility | 23.59 | 24.28 | 20.70 | 25.83 | 28.32 | 32.41 | 8.86 |

### Test Example 6

### X-Ray Diffraction (XRD) Analysis of Paclitaxel Cholesterol Complex

Test samples: paclitaxel, cholesterol, paclitaxel cholesterol complex (sample from Example 5), physical mixture of paclitaxel and cholesterol (molar ratio of 1:1).

Detection condition: Cu—K target, 40 kV of tube voltage, 200 MA of tube flow, diffraction range is 3°<2θ<60°.

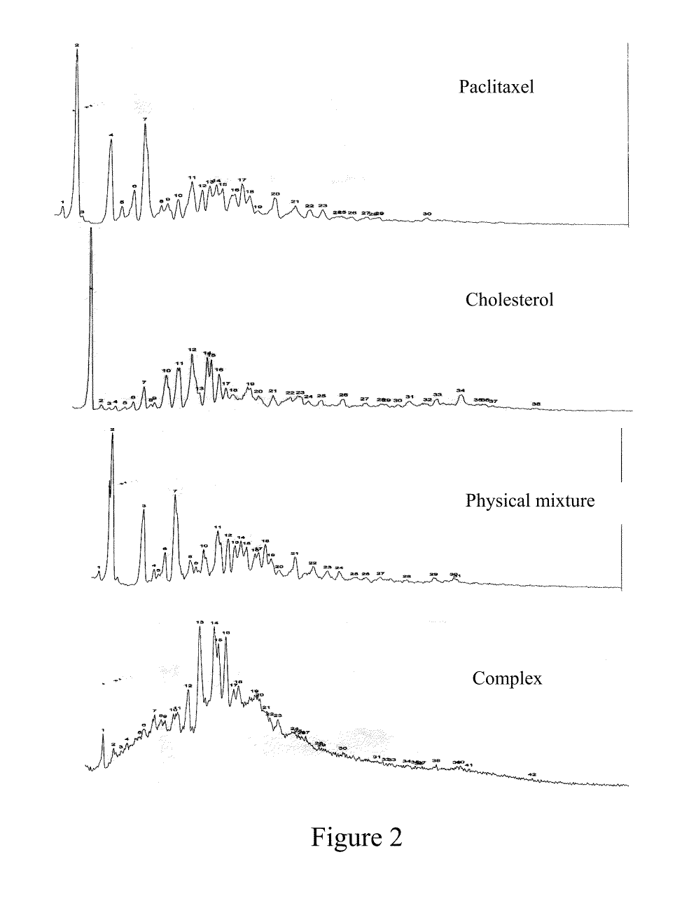

Results: diffraction results are shown in FIG. 2.

The results have clearly showed that the characteristic diffraction peaks of paclitaxel were in No. 2, 4, 7, the diffraction angle (2θ) was at 5.480, 8.840 and 12.180, respectively, the peak intensity was 23567, 11319, and 13277, respectively; the strongest diffraction peak of cholesterol was in No. 1, the diffraction angle (2θ) was at 5.160, the peak intensify was 35506; both characteristic diffraction peaks clearly appeared in the diffraction spectrum, that was the sum profile of paclitaxel and cholesterol profiles; but the diffraction spectrum of the complex changed. The characteristic diffraction peaks intensity of paclitaxel and cholesterol were greatly weaken or peaks disappeared, new characteristic diffraction peaks appeared at an angle of 15.240, 16.759, 17.160 and 17.960, respectively, the diffraction peak intensity was 4492, 3588, 2604 and 3186, respectively, lower than that of paclitaxel and cholesterol separately. This indicates that paclitaxel was dispersed at a microcrystalline or amorphous state in the complex.

### Test Example 7

### Infrared Spectrometry (IR) Analysis of Paclitaxel Cholesterol Complex

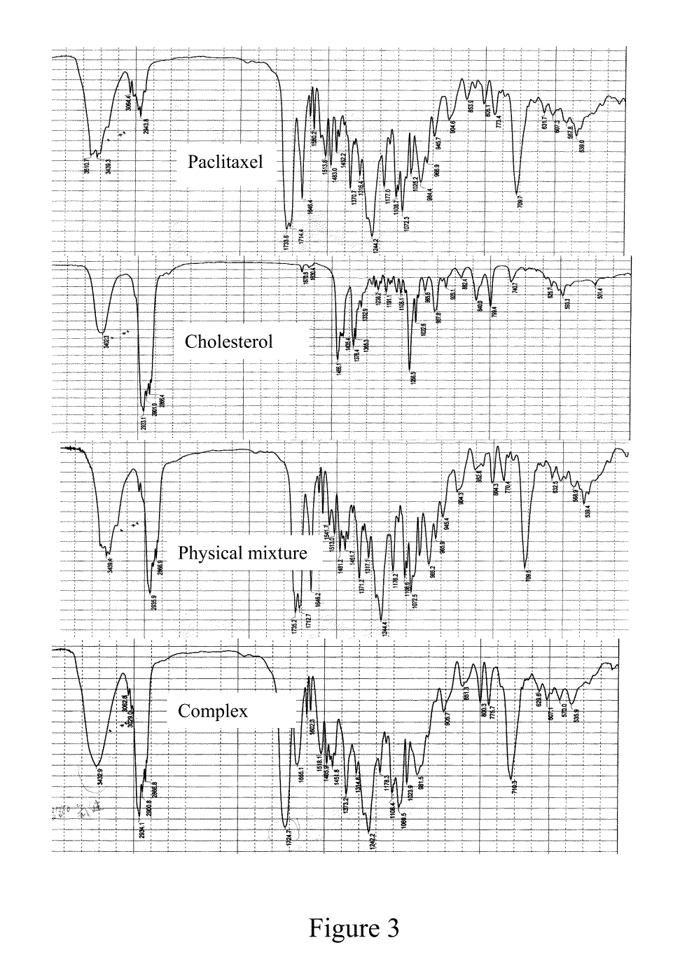

Take paclitaxel, cholesterol, a paclitaxel cholesterol complex respectively (sample with molar ratio of 1:1 from Example 5), a physical mixture of paclitaxel and cholesterol (with a molar ratio of 1:1), using potassium bromide to compress, carry out infrared spectroscopy method in the range of 400˜4000 cm-1, the results are shown in FIG. 3.

IR spectrum has shown that the main absorption peaks of paclitaxel were from the carbonyl group C=0 which had two splitting peaks at 1733.8 cm⁻¹ and 1714.4 cm⁻¹, the acylamino group which had the carbonyl peak at 1646.4 cm-1 as well as a stretching vibration absorption peak of the oxhydryl group O—H at 3300-3500 cm-1; in chloesterol IR sperum, the characteristic absorption peaks were the saturated C—H bonding strenching vibration peaks at 2933.1 cm-1, 2901.0 cm-1 and 2866.4 cm-1 and strenching vibration absorption peak of the oxhydryl group at 3402.3 cm-1; the IR specrum of the physical mixture was essentially the sum of absorption peaks obtained with cholesterol and paclitaxel; the characteristic absorption peaks of the complex changed, that is the characteristic peaks of the carbonyl group and acylamino group obtained in paclitaxel changed, two splitting peaks of the carbonyl group were converted into a blunt peak with strong absorption at 17247 cm-1, the peak shape of the carbonyl group in the acylamino group was blunted at 1646.4 cm-1; for cholesterol, the absorption peak shape of the oxhydryl group was widened at 3432.9 cm-1 in cholesterol and the absorption intensity was enhanced. These observations indicate that the carbonyl group of paclitaxel reacted with the oxhydryl group of cholesterol thereby producing a new complex.

### Test Example 8

### UV Absorption Spectroscopy (UV) and HPLC Chromatographic Peaks Study of Cholesterol Complex

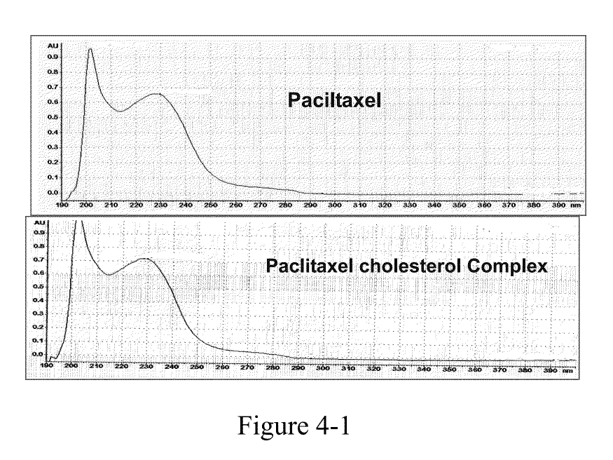

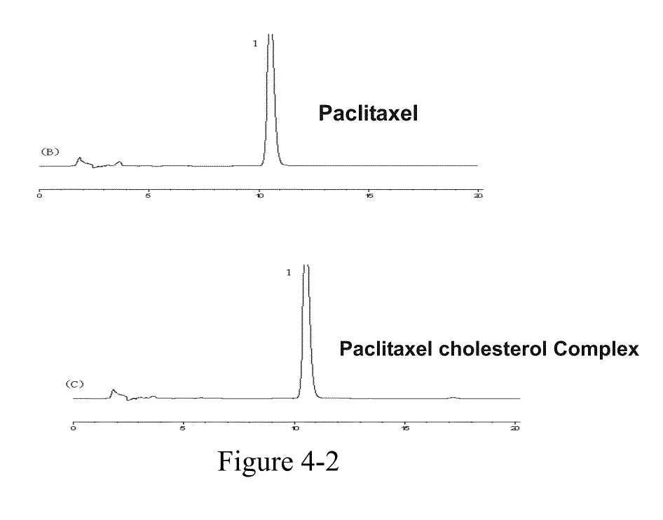

Dissolve paclitaxel and a paclitaxel cholesterol complex (sample with the molar ratio of 1:1 from Example 5) separately with absolute ethanol, using absolute ethanol as blank, carry out UV scanning method in the range of 200˜400 nm, the results are shown in FIG. 4-1. It is concluded that the UV characteristics of paclitaxel was consistent with that of the paclitaxel cholesterol complex, which indicates that the chromophore group of paclitaxel in the complex did not change, that is, the two components were combined to form a complex by intermolecular forces without forming new chemical bonds.

Inject 20 μl of ethanol solution of paclitaxel and paclitaxel cholesterol complex described above into the liquid chromatography, using Kromasil-C18 (250 mm×4.6 mm, 5 μm) as the column, acetonitrile-water (54:46) as the mobile phase, with a flow rate at 1.0 ml/min, a detection wavelength at 230 nm, record the retention time, and the results are shown in FIG. 4-2. It is concluded that the retention time of the main peak obtained with paclitaxel was consistent with that of the paclitaxel cholesterol complex, which indicates that the free paclitaxel can be ionized from the complex in a protonated ethanol solution.

### Test Example 9

### Stability Study of Paclitaxel Cholesterol Complex

Take solid powder of a drug steroid complex prepared from Example 1-18, store at 25° C., sampling periodically and examine the appearance changes. Add absolute ethanol into the solution at a suitable concentration, inject 20 μl into HPLC, using Kromasil C-18 (250 mm×4.6 mm, 5 μm) as the column, acetonitrile-water (54:46) as the mobile phase, with a flow rate at 1.0 ml/min, a detection wavelength at 230 nm, determine the contents and impurities. The results indicate that there was no significant change in the appearance, content or impurity, and the complex had stable quality compared to the original preparation prior to storage.

TABLE 6
Stability of paclitaxel steroid complex in long-term storage

| | | | | Degradated |
|---|---|---|---|---|
| Temperature/Time | | Appearance | Content (%) | products (%) |
| 25° C. | 0 month | White powder | 98.05~102.10 | ND |
| | 1 month | White powder | 98.25~102.23 | <0.2 |
| | 3 months | White powder | 97.78~101.74 | <0.3 |
| | 6 months | White powder | 98.90~102.36 | <0.3 |
| | 9 months | White powder | 98.55~101.76 | <0.2 |
| | 12 months | White powder | 98.36~101.87 | <0.2 |
| | 24 months | White powder | 98.71~101.88 | <0.2 |

### Test Example 10

### Test Example 10-1

### Paclitaxel Steroid Complex

Experimental complex 1-6: according to the technical requirements of the present invention, use cholesterol, 7-dehydrocholesterol and ergosterol to prepare the paclitaxel steroid complex with a molar ratio of 1:1-1:4. The detailed procedure is as follows: take paclitaxel and steroid into a triangle flask, dissolve with 2000 ml of acetone, mix at 40° C. for 1 hour, remove the solvent by rotary evaporation method, vacuum dry at 40° C. for 24 hours.

Reference complex 1-4: according to the technical requirements of Patent application CN200810168212.5, use soybean lecithin and cholesterol as the lipid materials to prepare 4 groups of reference complexes by the same method used for the comparative study, wherein the molar ratio in the paclitaxel phospholipids complex was 1:6 and 1:10, while the molar ratio in the paclitaxel cholesterol complex was 1:10 and 1:20, as detailed in the table below.

TABLE 7
Composition of paclitaxel cholesterol complex

| | | | Paclitaxel/lipid | |
|---|---|---|---|---|
| | | Amount of | materials | |
| Complex No./lipid | Amount of | lipid | Molar | |
| materials | paclitaxel | materials | ratio | Weight ratio |
| Experimental complex 1/ | 8.0 g | 3.6 g | 1:1 | 1:0.45 |
| cholesterol | | | | |
| Experimental complex 2/ | 8.0 g | 7.2 g | 1:2 | 1:0.90 |
| cholesterol | | | | |
| Experimental complex 3/ | 3.0 g | 1.35 g | 1:1 | 1:0.45 |
| 7-dehydrocholesterol | | | | |
| Experimental complex 4/ | 3.0 g | 5.406 g | 1:4 | 1:1.80 |
| 7-dehydrocholesterol | | | | |
| Experimental complex 5/ | 3.0 g | 1.40 g | 1:1 | 1:0.46 |
| ergosterol | | | | |
| Experimental complex 6/ | 3.0 g | 5.58 g | 1:4 | 1:1.86 |
| ergosterol | | | | |
| Reference complex 1/ | 3.0 g | 16.65 g | 1:6 | 1:5.55 |
| phospholipids | | | | |
| Reference complex 2/ | 3.0 g | 6.78 g | 1:10 | 1:9.23 |
| phospholipids | | | | |
| Reference complex 3/ | 3.0 g | 13.5 g | 1:10 | 1:4.50 |
| cholesterol | | | | |
| Reference complex 4/ | 3.0 g | 27.18 g | 1:20 | 1:9.06 |
| cholesterol | | | | |

### Test Example 10-2

### Paclitaxel Submicron Emulsion Prepared with Paclitaxel Cholesterol Complex as the Intermediate Carrier

[Formualtion]

| | submicron | | submicron | | submicron | | submicron | |
|---|---|---|---|---|---|---|---|---|
| Ingredients | emulsion 1 | | emulsion 2 | | emulsion 3 | | emulsion 4 | |
| Experimental | 145 | mg | 290 | mg | 580 | mg | 1160 | mg |
| complex 1* | | | | | | | | |
| Egg yolk | 2 | g | 2.4 | g | 3 | g | 3 | g |
| lecithin | | | | | | | | |
| Poloxamer | 1 | g | 2 | g | 4 | g | 6 | g |
| (188) | | | | | | | | |
| Glycerol | 5 | g | 5 | g | 5 | g | 5 | g |
| Soybean oil | 40 | ml | 40 | ml | 50 | ml | 50 | ml |
| Add water for | 200 | ml | 200 | ml | 200 | ml | 200 | ml |
| injection to | | | | | | | | |
| Total | 200 | ml | 200 | ml | 200 | ml | 200 | ml |

*Experimental complex 1 was the complex with a paclitaxel/cholesterol weight ratio of 1:0.45 prepared from Example 1.

[Preparation]
take 130-140 ml of water for injection, add egg yolk lecithin, Poloxamer (188) and glycerol according to the above formulation, transfer to a blender to disperse, obtain a homogeneous aqueous phase, heat to 40° C. and keep the temperature;
take a quantity of soybean oil according to the above formulation, preheat to 40° C. take paclitaxel cholesterol complex 1 prepared from Example 1, dissolve with preheated soybean oil, transfer to a blender to disperse, obtain a homogeneous oil phase;
add the aqueous phase slowly into the oil phase with constantly stirring, mix at 10,000 rpm/min for 5 mins, obtain a homogeneous colostrum, transfer quickly into high-pressure homogenizer, homogenizer 6 times, collect all of emulsion, adjusting pH with 0.1 mol/L of hydrochloric acid to 4.0±0.5, add water to 200 ml, shake and pack, sterilize at 115° C. for 30 minutes.

For submicron emulsion 1-4, the amount of emulsifier (egg yolk lecithin) was 1.0% (g/ml), 1.2% (g/ml), 1.5% (g/ml) and 1.5% (g/ml) of the total amount of the submicron emulsion, the amount of cosurfactant Poloxamer (188) was 0.5% (g/ml), 1.0% (g/ml), 2.0% (g/ml) and 3.0% (g/ml) of the total amount of the submicron emulsion, and the drug loading of paclitaxel was 0.5 mg/ml, 1.0 mg/ml, 2.0 mg/ml, 4.0 mg/ml. Determined by Laser particle size analyzer, the average particle size of 4 groups emulsion was 225 nm, 233 nm, 245 nm and 230 nm.

### Test Example 10-3

### Paclitaxel Submicron Emulsion Prepared with Paclitaxel Cholesterol Complex as the Intermediate Carrier

[Formualtion]

| | submicron | | submicron | | submicron | | submicron | |
|---|---|---|---|---|---|---|---|---|
| Ingredients | emulsion 5 | | emulsion 6 | | emulsion 7 | | emulsion 8 | |
| Experimental | 190 | mg | 380 | mg | 760 | mg | 1520 | mg |
| complex 2* | | | | | | | | |
| Egg yolk | 2 | g | 2.4 | g | 3 | g | 3 | g |
| lecithin | | | | | | | | |
| Poloxamer | 2.4 | g | 4 | g | 4 | g | 6 | g |
| (188) | | | | | | | | |
| Glycerol | 5 | g | 5 | g | 5 | g | 5 | g |
| Vitamin E | / | | / | | / | | 40 | mg |
| Soybean oil | 40 | ml | 40 | ml | 50 | ml | 50 | ml |
| Add water for | 200 | ml | 200 | ml | 200 | ml | 200 | ml |
| injection to | | | | | | | | |
| Total | 200 | ml | 200 | ml | 200 | ml | 200 | ml |

*Experimental complex 2 was the complex with a paclitaxel cholesterol complex weight ratio of 1:0.90 prepared from Example 1.

[Preparation]
take 130-140 ml of water for injection, add Poloxamer (188) and glycerol according to the formulation, transfer to a blender to disperse, obtaina homogeneous aqueous phase, heat to 80° C. and keep the temperature;
take a quantity of soybean oil according to formulation, preheat to 80° C., take complex 2, egg yolk lecithin and vitamin E according to the formulation, dissolve with preheated soybean oil, transfer to a blender to disperse, obtain a homogeneous oil phase;
add the aqueous phase slowly into the oil phase with constantly stirring, 20,000 rpm/min for 5 mins, obtain a homogeneous colostrum, transfer quickly into high-pressure homogenizer, homogenizer 6 times, collect all of emulsion, adjusting pH with 0.1 mol/L of hydrochloric acid to 5.5±0.5, add water to 200 ml, shake and pack, sterilize at 115° C. for 30 minutes.

For submicron emulsion 5-8, the amount of emulsifier (egg yolk lecithin) was 1.0% (g/ml), 1.2% (g/ml), 1.5% (g/ml) and 1.5% (g/ml) of the total amount of the submicron emulsion, the amount of cosurfactant Poloxamer (188) was 1.2% (g/ml), 2.0% (g/ml), 2.0% (g/ml) and 3.0% (g/ml) of the total amount of the submicron emulsion, and the drug loading of paclitaxel was 0.5 mg/ml, 1.0 mg/ml, 2.0 mg/ml, 4.0 mg/ml. Determined by Laser particle size analyzer, the average particle size of 4 groups emulsion was 246 nm, 262 nm, 231 nm, 242 nm.

### Test Example 10-4

### Paclitaxel Submicron Emulsion Prepared with Paclitaxel Cholesterol Complex as the Intermediate Carrier

[Formualtion]

| | submicron | | submicron | | submicron | | submicron | |
|---|---|---|---|---|---|---|---|---|
| Ingredients | emulsion 9 | | emulsion 10 | | emulsion 11 | | emulsion 12 | |
| Experimental | 145 | mg | 290 | mg | 580 | mg | 1450 | mg |
| complex 1* | | | | | | | | |
| Poloxamer | 4 | g | 4 | g | 4 | g | 4 | g |
| (188) | | | | | | | | |
| Glycerol | 5 | g | 5 | g | 5 | g | 5 | g |
| Vitamin E | / | | / | | / | | 40 | mg |
| MCO | 40 | ml | 40 | ml | 50 | ml | 50 | ml |
| Add water for | 200 | ml | 200 | ml | 200 | ml | 200 | ml |
| injection to | | | | | | | | |
| Total | 200 | ml | 200 | ml | 200 | ml | 200 | ml |

*Experimental complex 1 was the complex with a paclitaxel cholesterol weight ratio of 1:0.45 prepared from Example 1.
MCO: medium chain oil

[Preparation]

The same procedure as Test Example 10-3, wherein adjusting pH to 5.0±0.5.

For submicron emulsion 9-12, the amount of emulsifier (soybean lecithin) was 1.2% (g/ml), 1.2% (g/ml), 1.2% (g/ml) and 1.5% (g/ml) of the total amount of the submicron emulsion, the amount of cosurfactant Poloxamer (188) was 2.0% (g/ml) of the total amount of the submicron emulsion, and the drug loading of paclitaxel was 0.5 mg/ml, 1.0 mg/ml, 2.0 mg/ml and 5.0 mg/ml. Determined by Laser particle size analyzer, the average particle size of 4 groups emulsion was 165 nm, 153 nm, 127 nm, 138 nm.

### Test Example 10-5

### Paclitaxel Submicron Emulsion Prepared with Paclitaxel Cholesterol Complex as the Intermediate Carrier

[Formualtion]

| | submicron | | submicron | | submicron | | submicron | |
|---|---|---|---|---|---|---|---|---|
| Ingredients | emulsion 13 | | emulsion 14 | | emulsion 15 | | emulsion 16 | |
| Experimental | 290 | mg | 435 | mg | 580 | mg | 1450 | mg |
| complex 1* | | | | | | | | |
| soybean | 2.4 | g | 2.4 | g | 2.4 | g | 4.0 | g |
| lecithin | | | | | | | | |
| Poloxamer | 3 | g | 3 | g | 4 | g | 4 | g |
| (188) | | | | | | | | |
| Glycerol | 5 | g | 5 | g | 5 | g | 5 | g |
| Oil mixture** | 40 | ml | 40 | ml | 40 | ml | 50 | ml |
| Add water for | 200 | ml | 200 | ml | 200 | ml | 200 | ml |
| injection to | | | | | | | | |
| Total | 200 | ml | 200 | ml | 200 | ml | 200 | ml |

*Experimental complex 1 was the complex with a paclitaxel cholesterol weight ratio of 1:0.45 prepared from Example 1.
**Oil mixture was a mixture of soybean oil and medium chain oil (volume ratio of 1:1).

[Preparation]

The same procedure as Example 3, wherein adjusting pH to 4.5±0.5.

For submicron emulsion 13-16, the amount of the emulsifier (soybean lecithin) was 1.2% (g/ml), 1.2% (g/ml), 1.2% (g/ml) and 2.0% (g/ml) of the total amount of the submicron emulsion, the amount of cosurfactant Poloxamer (188) was 1.5% (g/ml), 1.5% (g/ml), 2.0% (g/ml) and 2.0% (g/ml) of the total amount of the submicron emulsion, and the drug loading of paclitaxel was 1.0 mg/ml, 1.5 mg/ml, 2.0 mg/ml and 5.0 mg/ml. Determined by Laser particle size analyzer, the average particle size of 4 groups emulsion was 145 nm, 138 nm, 133 nm, 146 nm.

### Test Example 10-6

### Paclitaxel Submicron Emulsion Prepared with Paclitaxel Cholesterol Complex as the Intermediate Carrier

[Formualtion]

| | submicron | | submicron | | submicron | | submicron | |
|---|---|---|---|---|---|---|---|---|
| Ingredients | emulsion 17 | | emulsion 18 | | emulsion 19 | | emulsion 20 | |
| Experimental | 190 | mg | 380 | mg | 760 | mg | 1520 | mg |
| complex 2* | | | | | | | | |
| egg yolk | 3.0 | g | 3.0 | g | 4.0 | g | 6.0 | g |
| lecithin | | | | | | | | |
| Poloxamer | 4 | g | 4 | g | 6 | g | 6 | g |
| (188) | | | | | | | | |
| Glycerol | 5 | g | 5 | g | 5 | g | 5 | g |
| Oil mixture** | 30 | ml | 40 | ml | 50 | ml | 60 | ml |
| Add water for | 200 | ml | 200 | ml | 200 | ml | 200 | ml |
| injection to | | | | | | | | |
| Total | 200 | ml | 200 | ml | 200 | ml | 200 | ml |

*Experimental complex 2 was the complex with a paclitaxel cholesterol weight ratio of 1:0.90 prepared from Example 1.
**Oil mixture was a mixture of soybean oil and MEDIUM CHAIN OIL (volume ratio of 1:1).

[Preparation]

The same procedure as Example 2, wherein adjusting pH to 5.5±0.5.

For submicron emulsion 17-20, the amount of the emulsifier (egg yolk lecithin) was 1.5% (g/ml), 1.5% (g/ml), 2.0% (g/ml) and 3.0% (g/ml) of the total amount of the submicron emulsion, the amount of cosurfactant Poloxamer (188) was 2.0% (g/ml), 2.0% (g/ml), 3.0% (g/ml) and 3.0% (g/ml) of the total amount of the submicron emulsion, and the drug loading of paclitaxel was 0.5 mg/ml, 1.0 mg/ml, 2.0 mg/ml and 5.0 mg/ml. Determined by Laser particle size analyzer, the average particle size of 4 groups emulsion was 255 nm, 263 nm, 285 nm, 232 nm.

### Test Example 10-7

### Paclitaxel Submicron Emulsion Prepared with Paclitaxel Cholesterol Complex as the Intermediate Carrier

[Formualtion]

| | submicron | | submicron | | submicron | | submicron | |
|---|---|---|---|---|---|---|---|---|
| Ingredients | emulsion 21 | | emulsion 22 | | emulsion 23 | | emulsion 24 | |
| Experimental | 145 | mg | 290 | mg | 580 | mg | 1160 | mg |
| complex 1* | | | | | | | | |
| Fatty glyceride | 3.0 | g | 4.0 | g | / | | / | |
| Polyoxy- | / | | / | | 4.0 | g | 6.0 | g |
| ethylene | | | | | | | | |
| sorbitan fatty | | | | | | | | |
| acid ester | | | | | | | | |
| Poloxamer | 3 | g | 4 | g | 4 | g | 6 | g |
| (188) | | | | | | | | |
| Glycerol | 5 | g | 5 | g | 5 | g | 5 | g |
| Oleic acid | 0.2 | g | 0.2 | g | 0.2 | g | 0.2 | g |
| Oil mixture** | 30 | ml | 40 | ml | 50 | ml | 60 | ml |
| Add water for | 200 | ml | 200 | ml | 200 | ml | 200 | ml |
| injection to | | | | | | | | |
| Total | 200 | ml | 200 | ml | 200 | ml | 200 | ml |

*Experimental complex 1 was the complex with a paclitaxel cholesterol weight ratio of 1:0.45 prepared from Example 1.
**Oil mixture was a mixture of soybean oil and medium chain oil (volume ratio of 1:1).

[Preparation]

The same procedure as Example 3

For submicron emulsion 21 and 22, the amount of the emulsifier (fatty glyceride) was 1.5% (g/ml) and 2.0% (g/ml) of the total amount of the submicron emulsion; for submicron emulsion 23 and 24, the amount of the emulsifier (polyoxyethylene sorbitan fatty acid ester) was 2.0% (g/ml) and 3.0% (g/ml) of the total amount of the submicron emulsion; for submicron emulsion 21-24, the amount of cosurfactant Poloxamer (188) was 1.5% (g/ml), 2.0% (g/ml), 2.0% (g/ml) and 3.0% (g/ml) of the total amount of the submicron emulsion, and the drug loading of paclitaxel was 0.5 mg/ml, 1.0 mg/ml, 2.0 mg/ml and 4.0 mg/ml. Determined by Laser particle size analyzer, the average particle size of 4 groups emulsion was 145 nm, 133 nm, 126 nm, 158 nm.

### Test Example 10-8

### Submicron Emulsion Prepared with 7-Dehydrocholesterol or Ergosterol as the Intermediate Carrier

[Formualtion]

| | submicron | | submicron | | submicron | | submicron | |
|---|---|---|---|---|---|---|---|---|
| Ingredients | emulsion 25 | | emulsion 26 | | emulsion 27 | | emulsion 28 | |
| Experimental | 290 | mg | / | | / | | / | |
| complex 3* | | | | | | | | |
| Experimental | / | | 5604 | mg | / | | / | |
| complex 4* | | | | | | | | |
| Experimental | / | | / | | 292 | mg | / | |
| complex 5* | | | | | | | | |
| Experimental | / | | / | | / | | 5720 | mg |
| complex 6* | | | | | | | | |
| Soybean | 2.4 | g | 2.4 | g | 2.4 | g | 4.0 | g |
| lecithin | | | | | | | | |
| Poloxamer | 3 | g | 3 | g | 4 | g | 4 | g |
| (188) | | | | | | | | |
| Glycerol | 5 | g | 5 | g | 5 | g | 5 | g |
| Oil mixture** | 40 | ml | 40 | ml | 40 | ml | 50 | ml |
| Add water for | 200 | ml | 200 | ml | 200 | ml | 200 | ml |
| injection to | | | | | | | | |
| Total | 200 | ml | 200 | ml | 200 | ml | 200 | ml |

*Experimental complex 3-6 was a mixture of the paclitaxel 7-dehydrocholesterol complex and the paclitaxel ergosterol complex prepared from Example 1.
**Oil mixture was a mixture of soybean oil and MEDIUM CHAIN OIL (volume ratio of 1:1).

[Preparation]

The same procedure as Example 5

For submicron emulsion 25 and 28, the drug loading of paclitaxel was 1.0 mg/ml. Determined by Laser particle size analyzer, the average particle size of 4 groups of emulsion was 143 nm, 138 nm, 141 nm, 132 nm.

### Test Example 10-9

### Paclitaxel Submicron Emulsion Prepared with the Reference Complex as the Intermediate Carrier

Take the reference complexes 1-4 prepared from Test Example 1, carry out the method with respect to the preparation of submicron emulsions as described in Example above, obtain submicron emulsions 29-32 with drug loading of 5, 1.0, 1.0 and 2.0 mg/ml, use for comparative study.

The results of the detailed formulation, preparation method and encapsulation efficiency are shown as follows:

[Formualtion]

| | submicron | | submicron | | submicron | | submicron | |
|---|---|---|---|---|---|---|---|---|
| Ingredients | emulsion 29 | | emulsion 30 | | emulsion 31 | | emulsion 32 | |
| Reference | 655 | mg | / | | / | | / | |
| complex 1* | | | | | | | | |
| Reference | / | | 1023 | mg | / | | / | |
| complex 2* | | | | | | | | |
| Reference | / | | / | | 1100 | mg | / | |
| complex 3* | | | | | | | | |
| Reference | / | | / | | / | | 4024 | mg |
| complex 4* | | | | | | | | |
| Egg yolk | 3 | g | 3 | g | 3 | g | 3 | g |
| lecithin | | | | | | | | |
| Poloxamer | 3 | g | 3 | g | 3 | g | 3 | g |
| (188) | | | | | | | | |
| Glycerol | 5 | g | 5 | g | 5 | g | 5 | g |
| Soybean oil | 40 | ml | 40 | ml | 50 | ml | 50 | ml |
| Add water for | 200 | ml | 200 | ml | 200 | ml | 200 | ml |
| injection to | | | | | | | | |
| Total | 200 | ml | 200 | ml | 200 | ml | 200 | ml |

*Reference complex 1 and 2 were two groups of reference complexes with a paclitaxel/phospholipids weight ratio of 1:5.55 and 1:9.23, respectively, prepared from Test Example 1.
*Reference complex 3 and 4 were two groups of the reference complexes with a cholesterol/paclitaxel phospholipids weight ratio of 1:4.50 and 1:9.06, respectively, prepared from Test Example 1.

[Preparation]
take 130-140 ml of water for injection, add egg yolk lecithin, Poloxamer (188) and glycerol according to the formulation, transfer to a blender to disperse, obtain a homogeneous aqueous phase, heat to 40-80° C. and keep the temperature;
take a quantity of soybean oil according to the formulation, preheat to 40-80° C., take paclitaxel cholesterol complex 1 prepared from Example 1, dissolve with preheated soybean oil, transfer to a blender to disperse, obtain a homogeneous oil phase;
add the aqueous phase slowly into the oil phase with constantly stirring, mix at 10,000-20,000 rpm/min for 5-10 mins, obtain homogeneous colostrum, transfer quickly into high-pressure homogenizer, homogenizer 6 times, collect all of emulsion, adjusting pH with 0.1 mol/L of hydrochloric acid to 4.5±0.5, add water to 200 ml, shake and pack, sterilize at 115° C. for 30 minutes.

The stability research and comparison of submicron emulsion prepared in Test Example s Above

Take 28 groups of submicron emulsions from Test Example s 10-2 to 10-8 and 4 groups of submicron emulsions from Test Example 10-9, store at 4° C. for 12 months respectively, sampling at 0, 6, 12 months, carry out the method as described below to examine the changes in appearance, particle size, content and impurity.

Characteristics: Visual examination, describe the color of the submicron emulsions, record whether oil droplets or phase separation appearson the surface.

Particle size: take the submicron emulsions, examine the particle size using MASTER SIZER 2000 laser particle size analyzer (malvem).

Contents and related substances: Take accurately the quantity of a paclitaxel submicron emulsion, add anhydrous ethanol to demulsification, obtain a suitable concentration of a test solution. Inject accurately 20 μl of the test solution into chromatography, carry out an HPLC analysis using Kromasil-C18(300 mm×4.6 mm, 5 μm) as the column, acetonitrile-water (54:46) as the mobile phase, with a flow rate of 1.0 ml/min, a detection wavelength of 230 nm, and the column temperature being the room temperature; record the chromatogram, calculate the content of the drug substance in each emulsion with respect to the peak by the external standard method, calculate the content of impurity by normalization method.

Results: as detailed in the table below

TABLE 8
Comparative results of submicron emulsion stability

| | Initial (examine within one week after | | | | | | | |
|---|---|---|---|---|---|---|---|---|
| Sample | preparation) | | | | Stored at 4° C. for 12 months | | | |
| No./drug | | Particle | | | | Particle | | |
| loading | Appearance | size | content | impurity | Appearance | size | content | impurity |
| Emulsion 1/ | Uniform | 225 nm | 100.2% | 0.33% | Uniform | 221 nm | 99.7% | 0.67% |
| 0.5 mg/ml | emulsion | | | | emulsion | | | |
| Emulsion 2/ | Uniform | 233 nm | 97.6% | 0.31% | Uniform | 245 nm | 97.3% | 0.62% |
| 1.0 mg/ml | emulsion | | | | emulsion | | | |
| Emulsion 3/ | Uniform | 245 nm | 98.5% | 0.31% | Uniform | 236 nm | 98.2% | 0.64% |
| 2.0 mg/ml | emulsion | | | | emulsion | | | |
| Emulsion 4/ | Uniform | 230 nm | 97.6% | 0.37% | Uniform | 237 nm | 97.0% | 0.93% |
| 3.0 mg/ml | emulsion | | | | emulsion | | | |
| Emulsion 5/ | Uniform | 246 nm | 99.5% | 0.35% | Uniform | 228 nm | 98.8% | 0.52% |
| 0.5 mg/ml | emulsion | | | | emulsion | | | |
| Emulsion 6/ | Uniform | 262 nm | 100.3% | 0.30% | Uniform | 255 nm | 99.5% | 0.61% |
| 1.0 mg/ml | emulsion | | | | emulsion | | | |
| Emulsion 7/ | Uniform | 231 nm | 98.1% | 0.36% | Uniform | 240 nm | 98.6% | 0.57% |
| 2.0 mg/ml | emulsion | | | | emulsion | | | |
| Emulsion 8/ | Uniform | 242 nm | 99.6% | 0.42% | Uniform | 251 nm | 97.3% | 1.01% |
| 4.0 mg/ml | emulsion | | | | emulsion | | | |
| Emulsion 9/ | Uniform | 165 nm | 98.8% | 0.35% | Uniform | 126 nm | 99.2% | 0.56% |
| 0.5 mg/ml | emulsion | | | | emulsion | | | |
| Emulsion 10/ | Uniform | 153 nm | 101.6% | 0.30% | Uniform | 133 nm | 100.7% | 0.52% |
| 1.0 mg/ml | emulsion | | | | emulsion | | | |
| Emulsion 11/ | Uniform | 127 nm | 102.2% | 0.32% | Uniform | 131 nm | 100.9% | 0.55% |
| 2.0 mg/ml | emulsion | | | | emulsion | | | |
| Emulsion 12/ | Uniform | 138 nm | 99.8% | 0.45% | Uniform | 155 nm | 99.4% | 1.24% |
| 5.0 mg/ml | emulsion | | | | emulsion | | | |
| Emulsion 13/ | Uniform | 145 nm | 97.4% | 0.31% | Uniform | 138 nm | 98.0% | 0.57% |
| 1.0 mg/ml | emulsion | | | | emulsion | | | |
| Emulsion 14/ | Uniform | 138 nm | 100.3% | 0.35% | Uniform | 136 nm | 98.7% | 0.65% |
| 1.5 mg/ml | emulsion | | | | emulsion | | | |
| Emulsion 15/ | Uniform | 133 nm | 99.2% | 0.31% | Uniform | 137 nm | 98.5% | 0.63% |
| 2.0 mg/ml | emulsion | | | | emulsion | | | |
| Emulsion 16/ | Uniform | 146 nm | 98.5% | 0.43% | Uniform | 173 nm | 97.2% | 1.68% |
| 5.0 mg/ml | emulsion | | | | emulsion | | | |
| Emulsion 17/ | Uniform | 255 nm | 97.8% | 0.32% | Uniform | 244 nm | 97.6% | 0.57% |
| 0.5 mg/ml | emulsion | | | | emulsion | | | |
| Emulsion 18/ | Uniform | 263 nm | 99.2% | 0.36% | Uniform | 259 nm | 99.5% | 0.53% |
| 1.0 mg/ml | emulsion | | | | emulsion | | | |
| Emulsion 19/ | Uniform | 285 nm | 100.4% | 0.33% | Uniform | 272 nm | 98.8% | 0.61% |
| 2.0 mg/ml | emulsion | | | | emulsion | | | |
| Emulsion 20/ | Uniform | 232 nm | 101.4% | 0.48% | Uniform | 258 nm | 97.8% | 1.76% |
| 5.0 mg/ml | emulsion | | | | emulsion | | | |
| Emulsion 21/ | Uniform | 145 nm | 98.7% | 0.36% | Uniform | 136 nm | 99.1% | 0.59% |
| 0.5 mg/ml | emulsion | | | | emulsion | | | |
| Emulsion 22/ | Uniform | 133 nm | 98.2% | 0.32% | Uniform | 127 nm | 97.6% | 0.57% |
| 1.0 mg/ml | emulsion | | | | emulsion | | | |
| Emulsion 23/ | Uniform | 126 nm | 101.1% | 0.38% | Uniform | 134 nm | 99.5% | 0.56% |
| 2.0 mg/ml | emulsion | | | | emulsion | | | |
| Emulsion 24/ | Uniform | 158 nm | 98.8% | 0.41% | Uniform | 163 nm | 97.9% | 1.25% |
| 4.0 mg/ml | emulsion | | | | emulsion | | | |
| Emulsion 25/ | Uniform | 143 nm | 99.5% | 0.33% | Uniform | 128 nm | 98.9% | 0.56% |
| 1.0 mg/ml | emulsion | | | | emulsion | | | |
| Emulsion 26/ | Uniform | 138 nm | 97.8% | 0.35% | Uniform | 132 nm | 97.4% | 0.58% |
| 1.0 mg/ml | emulsion | | | | emulsion | | | |
| Emulsion 27/ | Uniform | 141 nm | 99.4% | 0.30% | Uniform | 145 nm | 99.1% | 0.61% |
| 1.0 mg/ml | emulsion | | | | emulsion | | | |
| Emulsion 28/ | Uniform | 132 nm | 98.6% | 0.36% | Uniform | 126 nm | 98.2% | 0.54% |
| 1.0 mg/ml | emulsion | | | | emulsion | | | |
| Emulsion 29/ | Uniform | 253 nm | 97.5% | 0.92% | Layered with | 323 nm | 90.8% | 7.63% |
| 0.5 mg/ml | emulsion | | | | floading oil | | | |
| Emulsion 30/ | Layered | 522 nm | 93.2% | 6.27% | Oil-water | Not | Not | Not |
| 1.0 mg/ml | | | | | separation | detected | detected | detected |
| Emulsion 31/ | Uniform | 247 nm | 98.6% | 0.62% | Uniform | 263 nm | 94.7% | 3.58% |
| 1.0 mg/ml | emulsion | | | | emulsion | | | |
| Emulsion 32/ | Uniform | 266 nm | 98.9% | 0.78% | Slightly | 311 nm | 92.8% | 4.64% |
| 2.0 mg/ml | emulsion | | | | layered | | | |

Submicron emulsions 1-28 prepared with steroidal complexes as the intermediate carrier in the present invention, stored in the refrigerator (4° C.) for 12 months, were compared with the original preparations (prior to storage). 1) For emulsions with a 5.0 mg/ml drug loading, the average particle size appeared to have a trend to increase. However, the emulsions did not show layer, and the particle size, appearance and contents did not have obvious changes, the extent of impurity increased but did not exceed 2.0%; 2) emulsions with a 4.0 mg/ml drug loading did not show layer, and no obvious changes occurred in particle size, appearance or contents, impurity increased but did not exceed 1.3%; 3) emulsions with a 3 mg/ml drug loading did not show layer, and no obvious changes occurred in particle size, appearance or contents, impurity did not exceed 1.0%; 4) emulsions with a 2 mg/ml or less than 2 mg/ml drug loading did not have obvious changes in particle size, appearance or contents, impurity was less than 0.7%.

Submicron emulsions prepared with reference paclitaxel/phospholipids complexes as carrier: 1) a uniform emulsion (submicron emulsion 29) was formed with a drug loading of 0.5 mg/ml, and was kept for 6 months; there was no obvious change in appearance or particle size, and impurity increased to 3.0%, whereas if kept for 12 months, the particle size changed significantly and impurity increased to 7% or above, the content decreased with layered and floating oil; 2) if the drug loading was increased into 1.0 mg/ml, the uniform emulsion was not formed (submicron emulsion 30), the drug crystallization and oil droplets appeared at the beginning

Submicron emulsions prepared with reference paclitaxel/cholesterol complexes as carrier: 1) uniform emulsions (sample 31-32) were formed with a drug loading of 1.0 mg/ml and 2.0 mg/ml, respectively, and were kept for 6 months; there was no obvious change in appearance, particle size or contents, and impurity increased but did not exceed 1.5%; 2) if kept for 12 months, the particle size changed significantly, the drug content decreased, and impurity increased to 3.58% and 4.64%, wherein the emulsion with a drug loading of 2.0 mg/ml had slightly layered.

1. A paclitaxel steroid complex, wherein the complex is composed of paclitaxel and steroid with molar ratio of 1:0.2˜4.

2. The paclitaxel steroid complex according to claim 1, wherein the molar ratio of paclitaxel and steroid is 1:0.25˜2.

3. The paclitaxel steroid complex according to claim 2, wherein the molar ratio of paclitaxel and steroid is 1:0.33˜1.

4. The paclitaxel steroid complex according to claim 1, wherein said steroid is a natural steroid or a derivative thereof.

5. The paclitaxel steroid complex according to claim 4, wherein said natural steroid is selected from the group consisting of cholesterol, 7-dehydrocholesterol, lanosterol, sitosterol, brassicasterol, mycosterol, oysters steroid, stigmasterol, sitosterolum and ergosterol; said derivative is selected from the group consisting of cholic acid, deoxycholic acid and anthropodesoxycholic acid.

6. The paclitaxel steroid complex according to claim 1, wherein said complex contains an antioxidant stabilizer.

7. The paclitaxel steroid complex according to claim 6, wherein said antioxidant stabilizer is selected from the group consisting of sodium bisulfate, sodium metabisulfite, vitamin C, EDTA and its salts, and vitamin E and its derivatives.

8. A method for preparing a paclitaxel steroid complex according to claim 1 said method comprising the following steps:
a. mixing paclitaxel and steroid lipid materials in proportion, adding an organic solvent to dissolve, adding an antioxidant stabilizer; and
b. stirring at a suitable temperature, removing the organic solvent, and vacuum drying.

9. The preparation according to claim 8, wherein during step a), taking a quantity of paclitaxel and steroid, dissolving separately with different organic solvents, and mixing.

10. The method according to claim 8 or 9, wherein said organic solvent is selected from the group consisting of dichloromethane, ethanol, methanol, benzyl alcohol, acetone, ethyl acetate, tetrahydrofuran and tert-butanol.

11. The method according to claim 8 or 9, wherein said organic solvent is selected from the group consisting of ethanol, acetone, ethyl acetate and tetrahydrofuran.

12. Use of the paclitaxel steroid complex according to claim 1 for the preparation of an oil-in-water submicron emulsion, a dry emulsion, a self-microemulsifying system or oral preparations.
